(19) United States
(12) Patent Application Publication
(10) Pub. No.: US 2022/0323660 A1
(43) Pub. Date: Oct. 13, 2022

(54) MEDICAL TREATMENT DEVICES WITH CONTROLLABLE LIGHT GUIDES AND SMART COVERS

(71) Applicant: Fresenius Medical Care Holdings, Inc., Waltham, MA (US)

(72) Inventors: James Peterson, Benecia, CA (US); Stephen A. Merchant, Sudbury, MA (US); David Yuds, Hudson, NH (US); Jon F. Moss, Antioch, CA (US)

(21) Appl. No.: 17/224,486

(22) Filed: Apr. 7, 2021

(51) Int. Cl. A61M 1/16

(52) U.S. Cl. CPC A61M 1/1621; A61M 2205/587; A61M 2205/14; A61M 2205/18; A61M 2205/583; A61M 2205/52

(57) ABSTRACT

In one aspect, a medical device includes: a housing; a plurality of attachment points disposed on a face of the housing and configured to hold a medical fluid tubing system; a lighting system disposed at or below the face of the housing; and a processor configured to control the lighting system to (a) visually indicate the locations of the attachment points and (b) in response to a feedback signal indicating whether or not the medical fluid tubing system is attached to at least one of the attachment points, alter the visual indication of the lighting system.

### TECHNICAL FIELD

This disclosure relates to medical treatment devices, and more particularly to medical treatment devices having fluid tubes or other components that are manually mounted to the devices by users.

### BACKGROUND

Some medical treatments utilize machines to which various components (e.g., disposable components) are mounted for the machines to operate during a treatment procedure. For example, pumps used for IV fluids require the insertion of IV tubing lines or cassettes; apheresis machines require tubing and blood bags to be mounted in a specific arrangement; and many dialysis machines require an operator (e.g., clinician or patient) to mount one or more disposable blood line sets to the machine.

Dialysis is a treatment used to support a patient with insufficient renal function. The two principal dialysis methods are hemodialysis and peritoneal dialysis. During hemodialysis (“HD”), the patient's blood is passed through a dialyzer of a dialysis machine while also passing a dialysis solution or dialysate through the dialyzer. A semi-permeable membrane in the dialyzer separates the blood from the dialysate within the dialyzer and allows diffusion and osmosis exchanges to take place between the dialysate and the blood stream. These exchanges across the membrane result in the removal of waste products, including solutes like urea and creatinine, from the blood. These exchanges also regulate the levels of other substances, such as sodium and water, in the blood. In this way, the dialysis machine acts as an artificial kidney for cleansing the blood.

The blood flows from the patient to what is referred to as an extracorporeal blood circuit. This circuit is the pathway from the point that the blood exits the patient's body to the point where it is returned after dialysis has taken place. The extracorporeal circuit, which includes arterial and venous blood lines, as well as the dialyzer, is disposed of between treatments, and replaced with a new one.

In some systems, setting up the extracorporeal blood circuit for a hemodialysis treatment involves inserting and connecting almost a dozen feet worth of sterile tubing into a hemodialysis machine. This tubing is placed in tubing guides to prevent kinking. Moreover, the tubing must be correctly placed to interface with various pumps and sensors to allow for proper operation during treatment.

In an effort to correctly place the tubing on the hemodialysis machine, an operator typically refers to a diagram which is normally contained in a printed operator's manual or displayed on the screen of the dialysis machine. However, this arrangement is prone to error as the operator may overlook certain connections and either cause a delay to the treatment or negatively impact the therapy. In the above example, the bloodline paths are not linear or intuitive, making setup especially prone to error.

To minimize errors, some machines used for medical procedures show visual instructions on the machine display intended to aid the user during set up. These instructions can include diagrams, photos or animations. In a typical set up, the user completes one step, confirms the step (by pressing a button or other means) before the machine progresses to display the next step. While this approach is helpful, especially for novice users, the method can become tedious for experienced or expert users, who no longer need to stop or delay to confirm each step in the operation.

Ideally, when dialysis machines are used in a home setting, the machine should aid the user to minimize potential use errors, but not add additional steps which increase the time for set up or otherwise frustrate the user.

In a similar fashion, at the end of a medical treatment, the disposable set, blood lines, tubing, cassettes, etc., need to be removed from the machine before the system can be shut down or made available for the next patient or the next procedure. Typically, the operator's manual or the instructions on the machine's display aid the user in with step-by-step instructions.

Peritoneal dialysis (PD) devices may require routing tubing through clamps, pumps, flow sensors, temperature sensors, peritonitis/turbidity sensors, etc.

Other medical devices involving complex mounting of tubing present similar issues.

Moreover, medical equipment, such as dialysis machines, can negatively impact patient comfort and morale, especially in the home setting. In this regard, the visibility of pumps, clamps, fluid lines, and other aspects can stand out in a home setting and cause the environment to feel clinical.

### SUMMARY

This disclosure relates to medical treatment devices having fluid tubes or other components that are manually mounted to the devices by users and mechanisms to guide such mounting.

In one aspect, a medical device includes: a housing; a plurality of attachment points disposed on a face of the housing and configured to hold a medical fluid tubing system; a lighting system disposed at or below the face of the housing; and a processor configured to control the lighting system to (a) visually indicate the locations of the attachment points and (b) in response to a feedback signal indicating whether or not the medical fluid tubing system is attached to at least one of the attachment points, alter the visual indication of the lighting system.

The medical device may be a blood treatment device, such as a dialysis machine.

The lighting system may include a plurality of light-emitting diodes disposed below the face of the housing.

The face of the housing may include a translucent material configured to diffuse light emitted from the array of light-emitting diodes.

The feedback signal may correspond to a query response from a user of the medical device.

The medical device may further include a sensor system configured to generate the feedback signal using a plurality of sensors configured to monitor respective attachment points of the plurality of attachment points.

The processor may be configured to: determine, based on signals from the sensor system, that the medical fluid tubing system has been unseated or dismounted from one of the attachment points; and control the lighting system to visually indicate the location of the attachment point at which the unseating or dismounting has been determined.

The processor may be configured to cause one or more lights of the lighting system to illuminate in proximity to the location of the attachment point at which the unseating or dismounting has been determined.

The processor may be configured to illuminate portions of the lighting system based on the feedback signal and at least one of (a) a predetermined installation sequence and (b) a predetermined dismounting sequence.

The predetermined installation sequence or the predetermined dismounting sequence may include a predetermined order of attachment points to which the medical fluid tubing system is to be mounted or dismounted.

The processor may be further configured to: illuminate a first portion of the lighting system in proximity to a first attachment point; and responsive to the feedback signal indicating attachment of the medical fluid tubing system to the first attachment point, illuminating a second portion of the lighting system in proximity to a second attachment point, the second attachment point immediately following the first attachment point in the predetermined order of attachment points.

The processor may be further configured to, responsive to to the feedback signal indicating attachment of the medical fluid tubing system to the second attachment point, illuminate a third portion of the lighting system in proximity to a third attachment point, the third attachment point immediately following the second attachment point in the predetermined order of attachment points.

The processor may be further configured to: simultaneously illuminate the lights in proximity to each of the attachment points; and responsive to the feedback signal indicating attachment of the medical fluid tubing system to a first one of the attachment points, change the appearance of a portion of the lighting system in proximity to the first one of the attachment point.

The altering of the visual indication may include controlling a portion of the lighting system by at least one of: (a) changing light intensity of the portion, (b) powering off the portion, (c) changing the portion from a visually perceptible blinking state to a non-blinking state, and (d) changing a color of the portion.

The portion of the lighting system may be in proximity to at least one attachment point where attachment of the tubing system has been detected, and the controlling is in response to the detection.

In one aspect, a medical device includes: a housing configured to receive a medical fluid tubing set; a cover having an open position to provide user access to install and remove the blood tubing set, and a closed position to at least partially cover the blood tubing set; and a processor configured control the appearance of the cover based on a sensed condition of the device.

The medical device may be a blood treatment device, such as a dialysis machine, and medical fluid tubing set is a blood tubing set.

The processor may be configured to change the appearance of the cover from a first state, in which the cover is transparent, to a second state, in which the cover is translucent or opaque.

The sensed condition is whether the cover is in the open position or the closed position, and the processor may be configured to change the appearance of the cover from the first state to the second state in response to a determination that the cover in the closed position.

The processor may be configured to wait a period of time after sensing the closed position before changing the appearance from the first state to the second state.

The processor may be configured to change the appearance of the cover from the second state to the first state in response to an alarm condition of the blood treatment device.

The cover may include a transparent electronic display, such as a super transparent LED glass.

The processor may be configured to, in response to a determination of an alarm condition, display an alarm message on the cover.

The cover may include at least one hinged door.

In one aspect, a medical device includes: a housing; a light source configured to project light onto a surface adjacent to the housing; an image sensor configured to sense reflected portions of light projected by the light source; and a processor configured to (a) determine, based on the reflected portions of light sensed by the image sensor, whether the surface adjacent to the housing satisfies a predetermined size requirement, and (b) communicate the determination to a user of the device.

The medical device may be a blood treatment device such as a dialysis machine.

The medical device may further include a display screen, and the processor may be configured to, responsive to a determination that the surface satisfies the predetermined size requirement, control the display screen to display a message instructing the user to place an item on the surface.

The message may include an instruction to place the item on a visual indicator projected onto the surface by the light source.

The processor may be configured to change the light projected by the light source from a first visible color to a second color in response to a determination that the surface satisfies the predetermined size requirement.

Other aspects, features, and advantages will be apparent from the description, the drawings, and the claims.

### DESCRIPTION OF DRAWINGS

FIG. 1 is a front view of a blood treatment apparatus with a disposable cassette and blood line set, and unilluminated visual guides.

FIG. 2 is a front view of the blood treatment apparatus of FIG. 1, without the disposable cassette and blood line set, with visual guides illuminated.

FIGS. 3A to 3G show a portion of the blood treatment apparatus of FIG. 1 with the visual guides in respective sequential states corresponding to a blood line attachment procedure.

FIGS. 4A to 4G show a portion of the blood treatment apparatus of FIG. 1 with the visual guides in respective sequential states corresponding to a blood line attachment procedure.

FIG. 5 shows a cross section of a visual indicator having an array of indicator lights.

FIG. 6 shows a front view of the visual indicator of FIG. 5 with the translucent panel omitted to facilitate illustration.

FIG. 7 shows a front view of the visual indicator of FIG. 5 with light from the indicator lights blended by diffusion by way of the translucent panel to show an illuminated visual pattern.

FIG. 8 shows a front view of a visual indicator having an array of indicator lights with a translucent panel omitted to facilitate illustration.

FIG. 9 shows the visual indicator of FIG. 8 with light from the indicator lights blended by diffusion by way of the translucent panel to show an illuminated visual pattern.

FIG. 10 shows a front view of a blood treatment apparatus with doors in an open position.

FIG. 11 shows the blood treatment apparatus of FIG. 10 with the doors in a closed orientation and in a transparent state.

FIG. 12 shows the blood treatment apparatus of FIG. 10 with the doors in a closed orientation and in a transparent state with an electroluminescent wire illuminated.

FIG. 13 shows the blood treatment apparatus of FIG. 10 with the doors in a closed orientation and in an opaque state.

FIG. 14 shows the blood treatment apparatus of FIG. 10 with the doors in a closed orientation, in a transparent state, and using a transparent electronic display to show visual indicator graphics and text responsive to a detected blood line disconnection.

FIG. 15 shows the blood treatment apparatus of FIG. 10 with the doors in a closed orientation, in a transparent state, and using the transparent electronic display to show visual indicator graphics and text responsive to a detected blood leak.

FIG. 16 shows a blood treatment apparatus positioned on a support surface to provide insufficient adjacent surface space for supplies or containers.

FIG. 17 shows the blood treatment apparatus positioned on the support surface to provide sufficient adjacent surface space for the supplies or containers.

FIG. 18 shows a block diagram of an example computer system.

Like reference symbols in the various drawings indicate like elements.

### DETAILED DESCRIPTION

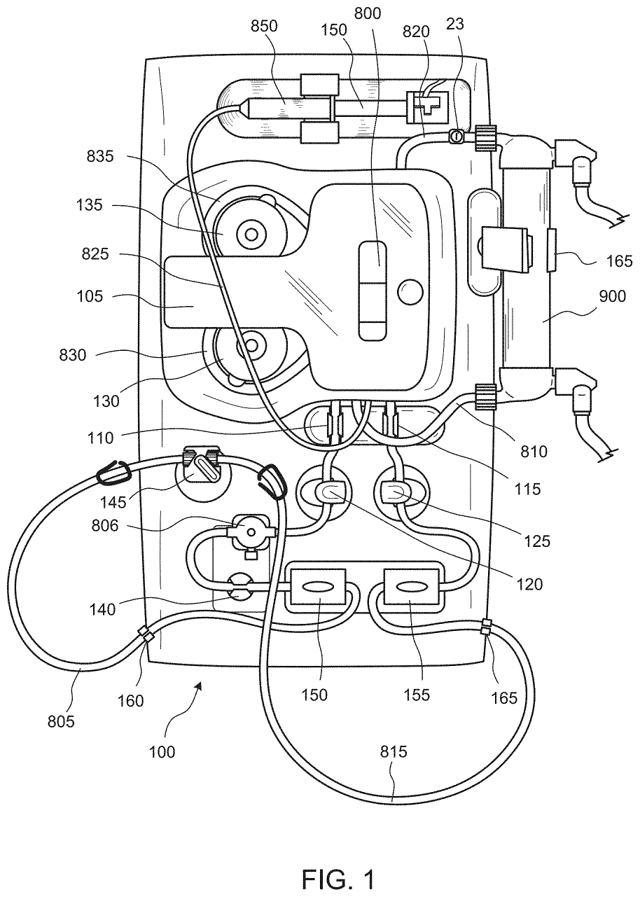

FIG. 1 shows a blood treatment apparatus for treating the blood of a patient. In this example, the blood treatment apparatus is a dialysis machine 100, which utilizes a disposable blood cassette 800 with fluid lines that are attached to the face of the dialysis machine 100 prior to beginning a patient treatment. These lines include a patient-side arterial blood line 805, a dialyzer-side arterial blood line 810, a patient-side venous blood line 815, a dialyzer-side venous blood line 820, and a heparin line 825. Also extending from the body of the cassette are two sections of pump tubing 830 and 835 that engage with respective rotors 130 and 135 of the dialysis machine 100 to form respective peristaltic pumps. It will be appreciated that the descriptions of the components are for example only and the invention is not dependent upon particular components in the illustrated machine; i.e., these pumps may take the form of piston pumps or the pump heads themselves may be disposable and part of the cassette, utilizing a pumping system like Quantex single-use pumps (https://www.quantex-arc.com/).

During setup of the machine 100, a cover 105 on the face of the machine is opened to allow the cassette 800 to be mounted to the machine. The cassette 800 snaps into place with the two sections of pump tubing 830 and 835 mating to the rotors 130 and 135. At this stage, the various tubes extending from the cassette 800 are unsecured and hanging loosely from the face of the machine 100. The person setting up the machine then positions and secures the various tubes to their appropriate locations on the machine.

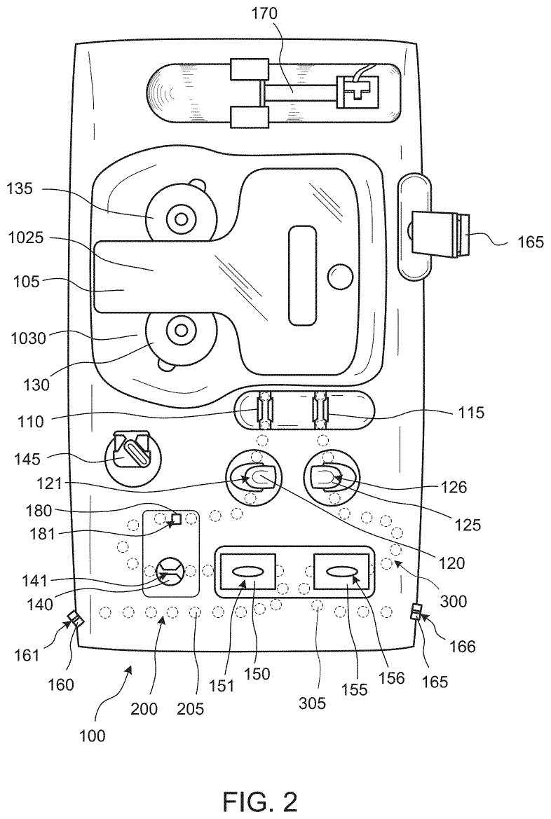

The patient-side arterial blood line 805 is pressed into an optical sensor 110 for detecting arterial air bubbles and/or the presence of liquid in the line 805. The line 805 is next pressed into an arterial clamp 120 for automated occlusion of the blood line 805 by the machine 100, and then bent toward a pressure pod port 180 (visible in FIG. 2), to which a pressure pod 806 is attached to allow the machine 100 to measure arterial blood pressure.

The line 805 is next bent into to form a U shape portion that is pressed into a line holder 140. The line 805 is then mounted to an arterial blood temperature monitor 150 for monitoring of the temperature of the blood in the line 805 by the machine 100. The line 805 is then bent back roughly 180 degrees to extend in the opposite direction and into another line holder 160. In this example, the patient-side arterial blood line 805 is then coupled to a recirculation connection 145 for priming.

The patient-side venous blood line 815 is mounted in a manner similar to that described above with respect to arterial line 805. The patient-side venous blood line 815 is pressed into an optical sensor 115 for detecting venous air bubbles and/or the presence of liquid in the line 815. The line 815 is next pressed into a venous clamp 125 for automated occlusion of the blood line 815 by the machine 100.

The line 815 is next bent into to form a U shape portion that is mounted to a venous blood temperature monitor 155 for monitoring of the temperature of the blood in the line 815 by the machine 100. The line 815 is then bent back roughly 180 degrees to extend in the opposite direction and into a line holder 165. In this example, the patient-side venous blood line 815 is then coupled to the recirculation connection 145 to allow flow between the respective ends of the patient-side arterial and venous tubes 805 and 815 for recirculating flow during priming.

The setup process also includes securing the dialyzer-side arterial and venous blood lines 810 and 820 to respective ends of a dialyzer 900 mounted in a clamp 165 of the dialysis machine 100. The process further includes mounting a heparin syringe 850 in a heparin pump 170 of the machine 100 with the mounted heparin line 825 extending to the outlet of the syringe 850.

During the setup of the machine 100, the mounting of the various disposable lines to the machine 100 can be confusing, especially the patient-side arterial and venous blood lines 805 and 815 with their multiple bends and connection points.

Referring to FIG. 2, to facilitate proper mounting of the disposable lines, the machine 100 includes visual guides 200 and 300, each in the form of a series of lights 205, 305 displaying through the face of the machine 100. In this example, the guides 200 and 300 are not visible when not activated. That is, the front surface of the machine 100 at the location of the guides 200 and 300 blends in with the surrounding area of the front surface when the lights 205 and 305 of the guides 200 and 300 are not illuminated. This may be achieved, for example, by making the front surface, or portions thereof sufficiently translucent to allow visibility of the lights when illuminated.

The machine 100 further employs a plurality of sensors to detect whether the tubing 805, 815 is properly connected at various respective locations. Following the order of attachment points for the arterial blood line 805, the machine 100 utilizes the optical sensor 110, a sensor 121 at the arterial clamp 120, a sensor 181 at the pressure pod port 180, a sensor 141 at the line holder 140, a sensor 151 at the arterial blood temperature monitor 150, and a sensor 161 at the line holder 160. Following the order of attachment points for the venous blood line 815, the machine 100 utilizes the optical sensor 115, a sensor 126 at the venous clamp 125, a sensor 156 at the venous blood temperature monitor 155, and a sensor 166 at the line holder 165 to detect proper connection.

Accordingly, the machine 100 provides a network of sensors and lights (e.g., LEDs) that can detect the presence of the tubing and illuminate accordingly. As an example procedure, when the operator of the hemodialysis machine 100 is prompted to string the arterial bloodline 805, the machine 100 pulses the indicator lights 205 (e.g., red LEDs) sequentially in the path the bloodline takes descending from the top, moving to the patient end of the bloodline 805. Along the way, sensors 110, 121, 181, 141, 151, and 161 detect the presence of the tubing of blood line 805 and, upon detection, the hemodialysis machine turns off the indicator lights 205 (and accordingly the corresponding portions of visual guide 200) leading to that sensor to indicate the tubing has been correctly attached. After the arterial bloodline 805 has been strung, the indicator lights 305 (e.g., blue LEDs) for the venous blood line 815 would then illuminate in an analogous process. In some examples, if a sensor detects tubing attachment but the immediately preceding sensor does not, the indicator lights around the bypassed sensor flash to draw attention to the omission until the tubing 805, 815 is properly inserted. Likewise, in some examples, if any tubing portion becomes dismounted during or after the setup procedure, the indicator lights around the relevant sensor flash or otherwise draw attention to the location of the dismount.

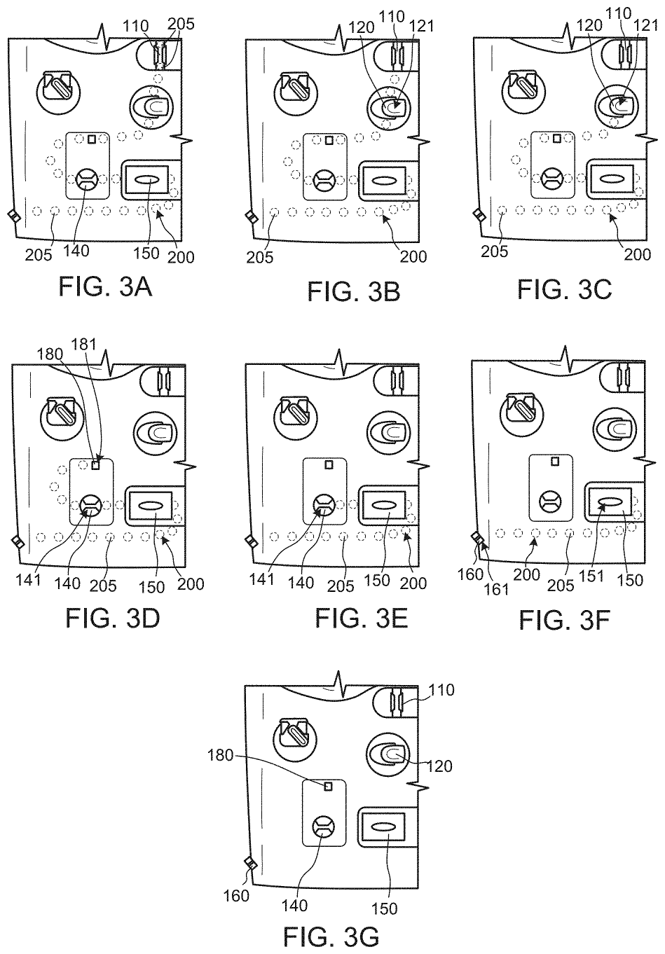

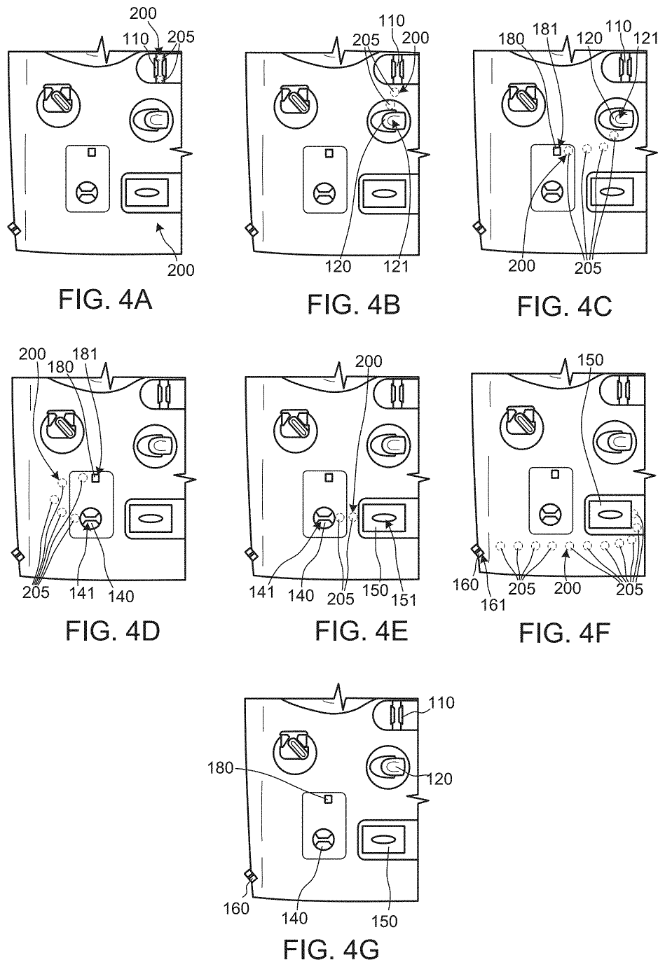

FIGS. 3A to 3G and FIGS. 4A to 4G show two different ways to illuminate the lights 205 of the visual guide 200 during mounting of the arterial blood line 805 after mounting the blood cassette 800 to the machine 100 and closing the cover 105. To facilitate illustration of the visual guide 200, the blood line 805 is not shown in FIGS. 3A to 3G and 4A to 4G.

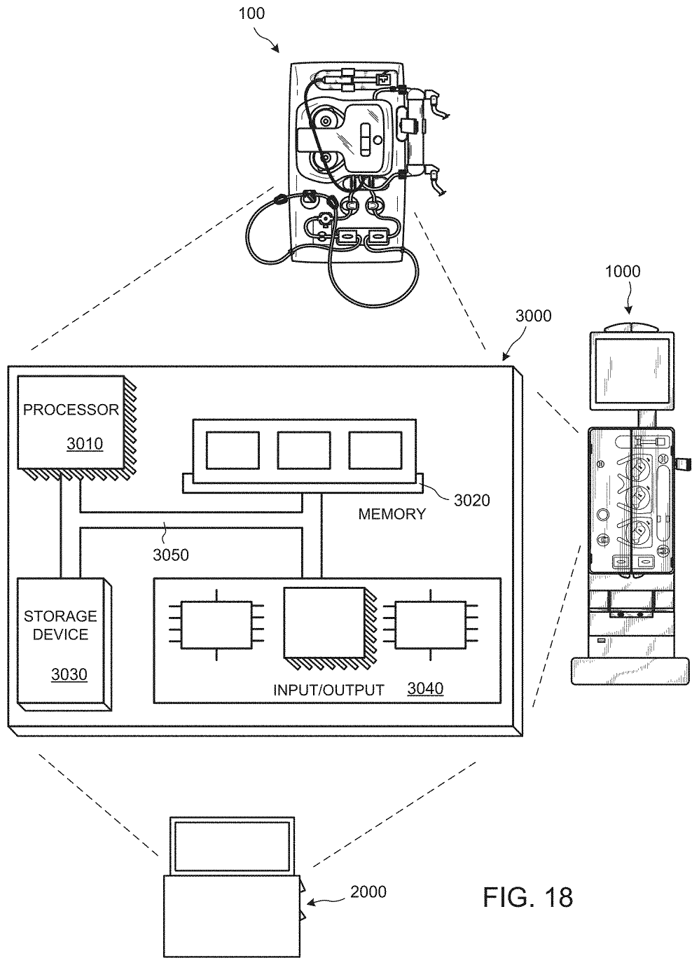

Referring to FIGS. 3A to 3G, the sequence of illumination of the visual guide 200 between six different lighting states is shown. In the first state, as shown in FIG. 3A, all of the lights 205 are illuminated to show the entire path of the blood line 805 as it is to mounted. In this example, this state of the guide 200 is shown after the operator is prompted (e.g., by a display or audio source) by the machine 100 to begin mounting the blood line 805. The operator would first press the blood line 805 into the arterial optical sensor 110. The optical sensor 110 would then convey signals to a processor (e.g., processor 3010 of system 3000 described below in connection with FIG. 18) of the machine 100 to indicate that the blood line 805 is seated in the optical sensor 110. In response to the indication from the optical sensor 110, the processor turns off the lights 205 along the portion of the guide 200 at the optical sensor 110 while leaving the remaining lights 205 of the guide 200 illuminated, as illustrated in FIG. 3B.

The operator then continues to place the blood line 805 along the path of the visual guide 200 by pressing the blood line 805 into the arterial clamp 120. In response to the mounting of the blood line 805 in the arterial clamp 120, the sensor 121 at the arterial clamp 120 sends a signal to the processor of the machine 100 to indicate that the mounting has occurred. In response to the indication from the sensor 121, the computer system turns off the lights 205 along the portion of the guide 200 that extends between the optical sensor 110 and the arterial clamp 120, leaving only the portions of the guide 200 that correspond to the portions of the blood line 805 that still need to be mounted.

The operator then continues to sequentially mount the blood line 805 in analogous manner, with mounting at each successive location (pressure pod port 180, line holder 140, arterial blood temperature monitor 150, and line holder 160) causing respective sensors 181, 141, 151, and 161 to transmit signals indicating successful mounting at respective components 180, 140, 150, and 160. In turn, and in response to the signals, the processor of the machine 100 turns off the lights 205 of the respective portions of the visual guide 200 immediately preceding the respective mounting locations 180, 140, 150, and 160 sequentially as the blood line 805 is installed along the path of the visual guide 200. In this regard, FIG. 3D shows the state of the visual guide 200 after the pressure pod 806 of the blood line 805 is mounted to the pressure pod port 180, FIG. 3E shows the state of the visual guide 200 after the blood line 805 is mounted in the line holder 140, FIG. 3F shows the state of the visual guide 200 after the blood line 805 is mounted in the arterial blood temperature monitor 150, and FIG. 3G shows the state of the visual guide 200 after the blood line 805 is mounted in the line holder 160.

In this example, as the illumination of each portion of the visual guide 200 is turned off, that respective portion of the visual guide is no longer visible on the face of the machine. As shown in FIG. 4G no part of the visual guide 200 is visible, as would be the case before the mounting procedure is initiated and after the blood line 805 is completed mounted.

Referring to FIGS. 4A to 4G, the sequence of illumination of the visual guide 300 between six different lighting states is shown. This sequence differs from that of FIGS. 3A to 3G in that only the portion of the visual guide 300 leading to the immediately preceding mounting location and the next sequential mounting location is illuminated to guide the user at each mounting step.

In the first state, as shown in FIG. 4A, only the lights 205 at the location of the optical sensor 110 are illuminated to direct the operator to secure the arterial blood line 805 to the optical sensor 110. In this example, this state of the guide 200 is shown after the operator is prompted (e.g., by a display or audio source) by the machine 100 to begin mounting the blood line 805. Upon the operator pressing the blood line 805 into the arterial optical sensor 110, the optical sensor 110 conveys signals to the processor of the machine 100 to indicate that the blood line 805 is seated in the optical sensor 110. In response to the indication from the optical sensor 110, the processor turns off the lights 205 at the optical sensor 110, and illuminates lights 205 along the path of the guide 200 between the optical sensor 110 while leaving the remaining lights 205 of the guide 200 illuminated, as illustrated in FIG. 3B.

The operator then continues to place the blood line 805 along the path of the visual guide 200 by pressing the blood line 805 into the arterial clamp 120. In response to the mounting of the blood line 805 in the arterial clamp 120, the sensor 121 at the arterial clamp 120 sends a signal to the processor of the machine 100 to indicate that the mounting has occurred. In response to the indication from the sensor 121, the processor turns off the lights 205 along the portion of the guide 200 that extends between the optical sensor 110 and the arterial clamp 120, leaving only the portions of the guide 200 that correspond to the portions of the blood line 805 that still need to be mounted.

The operator then continues to sequentially mount the blood line 805 in analogous manner, with mounting at each successive location (pressure pod port 180, line holder 140, arterial blood temperature monitor 150, and line holder 160) causing respective sensors 181, 141, 151, and 161 to transmit signals indicating successful mounting at respective components 180, 140, 150, and 160. In turn, and in response to the signals, the processor of the machine 100 turns off the lights 205 of the respective portions of the visual guide 200 immediately preceding the respective mounting locations 180, 140, 150, and 160 sequentially as the blood line 805 is installed along the path of the visual guide 200. In this regard, FIG. 3D shows the state of the visual guide 200 after the pressure pod 806 of the blood line 805 is mounted to the pressure pod port 180, FIG. 3E shows the state of the visual guide 200 after the blood line 805 is mounted in the line holder 140, FIG. 3F shows the state of the visual guide 200 after the blood line 805 is mounted in the arterial blood temperature monitor 150, and FIG. 3G shows the state of the visual guide 200 after the blood line 805 is mounted in the line holder 160.

In this example, as the illumination of each portion of the visual guide 200 is turned off, that respective portion of the visual guide is no longer visible on the face of the machine. As shown in FIG. 4G no part of the visual guide 200 is visible, as would be the case before the mounting procedure is initiated and after the blood line 805 is completed mounted. When the machine's programmed therapy is delivered, the system of lighted guides may turn on again to indicate the proper method to remove the tubing from the machine.

Similarly, the user may follow these type of light-indicated prompts for Peritoneal dialysis (PD) devices to insert or remove a cassette or cartridge and ensure each of the tubing lines to and from various dialysate solution bags has been properly placed. Other connection points may include routing tubing through clamps, pumps, flow sensors, temperature sensors, peritonitis/turbidity sensors, connector sterilizing devices like PuraCath's Firefly device (http.//puracath.com/), and sterile connection facilitation devices like the PeriSafe device (https://www.peripal.com/).

As indicated above, the indicator lights 205, 305 may be include lights of differing different colors to differentiate attachment of different components. For example, as suggested above, the indicator lights 205 for the arterial blood line 805 may be red when illuminated, and the indicator lights 305 for the venous blood line 815 may be blue when illuminated. In some examples, the mounted components themselves have at least one marking of the same color in order to further help the operator match each component to its respective path. For example, and expanding on the example above, the arterial line 805 may have at least one red marking to allow the user to associate the line 805 with path marked by the red indicator lights 205, and the venous line 815 may have at least one blue marking to allow the user to associate the line 815 with the blue indicator lights 305.

Moreover, other indicator light colors (e.g., green or yellow) could indicate additional tubing connections such as, for example, heparin lines, HDF tubing, dialyzers, and acid and bicarbonate concentrates (e.g. the connector for a bicarbonate or acid concentrate would illuminate when it is time to connect the concentrate container).

In accordance with the illustrated example, when not illuminated, all the indicator lights 205, 305 would appear hidden below the surface of the machine cabinet. In addition to preserving the appearance of the facade of the machine, this may also reduce the mental fatigue on the operator by reducing or eliminated the visual clutter of pathways that are always visible, even if not illuminated.

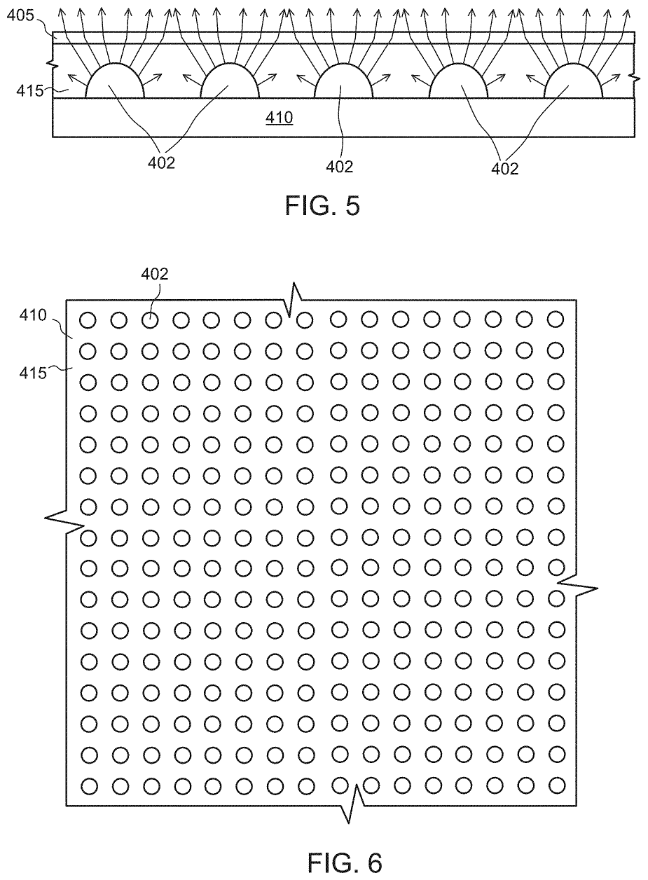

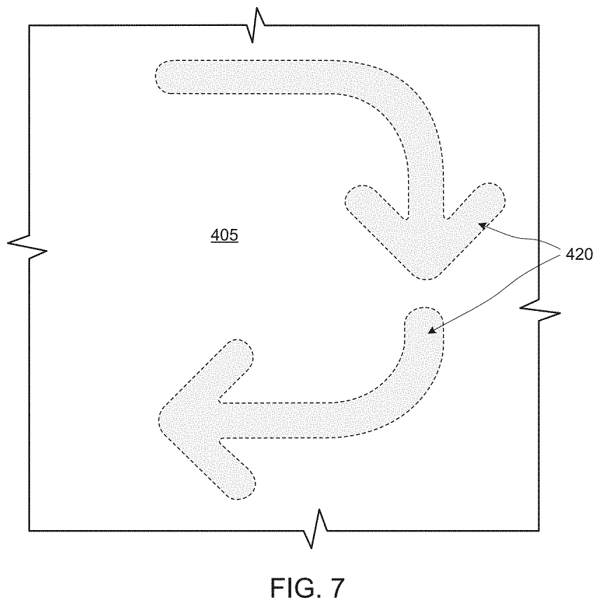

Referring to FIGS. 5 and 6, in some examples, the visual indicators are provided by an array of indicator lights 402 (e.g., LEDs, in some examples including multiple colors as discussed above or otherwise configured to emit a range of different colors) behind a diffused translucent panel 405. In this example, the lights 402, in the form of LEDs, are mounted on and controlled via a PC board 410. In the remainder of the space between the PC board 410 and the translucent panel 405 is a transparent layer 415, such as acrylic or any other suitable optical material. As shown in FIG. 5, when the lights 402 are illuminated they emanate light as shown by the arrows. This light travels through the transparent layer 415 and through the translucent panel 405, which functions to diffuse the light. In this manner, the light from adjacent lights 402 blend together to present as a continuous region of light on the face of the machine. FIG. 7 shows an example of this blended light, showing two curved arrows 420. Moreover, the translucent panel 405 acts to partially or completely prevent the lights 402 from being viewable by the operator when the lights 402 are not illuminated. In FIG. 6, the translucent panel 405 is omitted to facilitate illustration of the array of lights 402.

This array of lights 402 may be controlled, based on a computer program (executed, for example, as part of the computer system 3000 described below in connection with FIG. 18), to light up the specific blood line paths and in various colors. Moreover, the array of lights may be operated to morph into illuminated arrows, running text, or other visual queues.

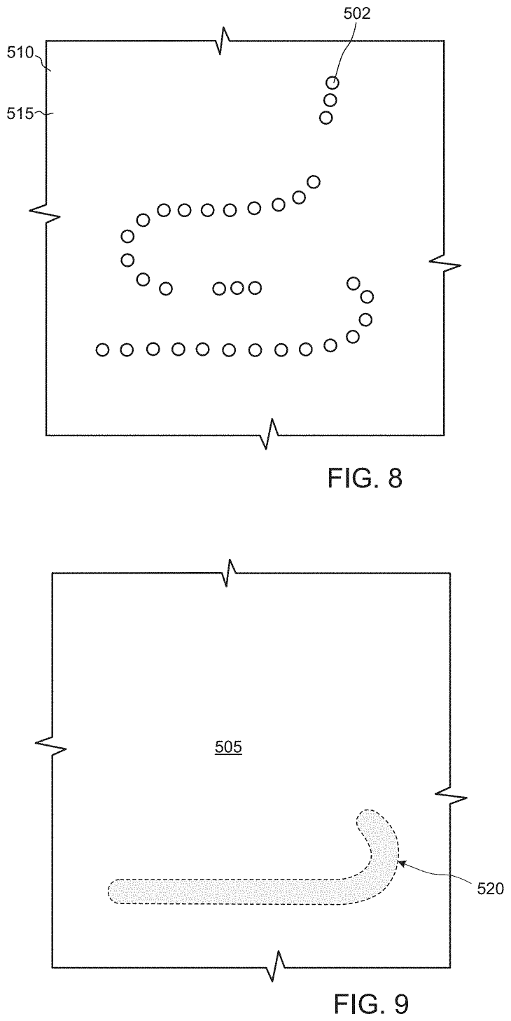

FIGS. 8 and 9 show a configuration similar to that of FIGS. 5 to 7. In this example, the indicator structure is formed with indicator lights 502 behind a diffused translucent panel 505. The lights 502 are mounted on and controlled via a PC board 510 with a transparent layer 515 disposed in the remaining space between the PC board 510 and the translucent panel 505. This example is constructed in the same manner as shown in the example of FIG. 5. However, comparing FIG. 6 and FIG. 8, the latter configuration differs in that it includes a less than a full array of lights. In this particular example, the lights 502 are only present in locations that correlate to the indication sequences of FIGS. 3A to 3G and 4A to 4G. As with FIG. 6 in the prior example, FIG. 8 omits the translucent panel 505 to facilitate illustration of the array of lights 502. FIG. 9 shows the light from adjacent lights 502 blending together via light diffusion to present as a continuous region of light on the face of the machine to show an illuminated line 520 indicating the location of tube attachment to an operator.

The tubing components described above, including, for example, the blood cassette 800, the venous and arterial blood lines 805, 810, 815, and 820, heparin line 825, and pump tubing 830 and 835 are part of a medical fluid tubing system. It should be understood that these components are part of the example used here for illustrative purposes, and that the concepts described herein may be likewise or analogously applied to other medical fluid tubing systems having different components and configurations than the examples described herein.

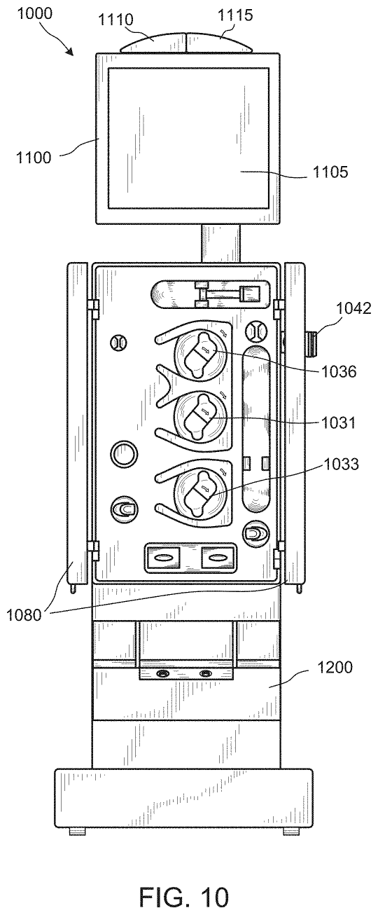

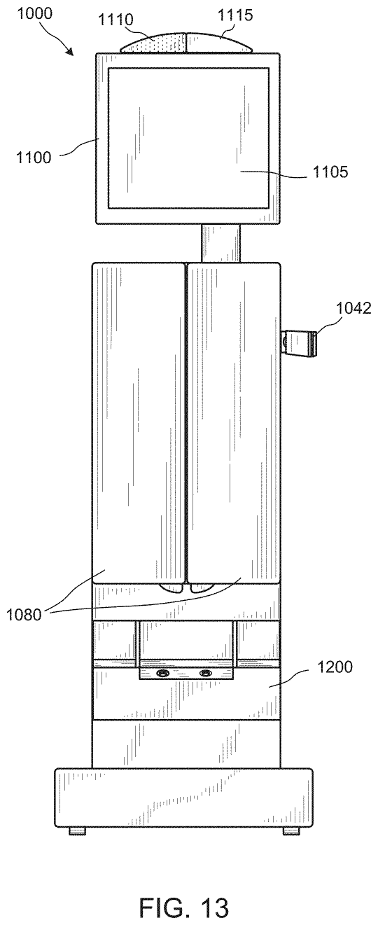

FIGS. 10 to 15 show another implementation of visual guidance and appearance control of a blood treatment apparatus, in the form of dialysis machine 1000. Such implementation may be utilized, for example, in home or clinical settings. Many patients, especially in the home treatment setting, prefer not to see the blood lines, pumps or other apparatus mechanisms; they would prefer the medical machine to look more like something that fits in a home setting rather than a hospital setting. However, there are times at set-up or during the operation of the machine when it would be helpful to have full visibility to the dialysis process to ensure the process is proceeding as planned or to better understand machine warnings or alarm conditions displayed by the machine.

Some dialysis machines specifically designed for home treatment have tried to address this problem by having all the mechanisms exposed at set up, but closed behind doors during operation. While this approach may address aesthetic concerns, if warnings, alarms or other issues arise during treatment, the patient or caregiver needs to open the doors to check the operation of the machine. This is not ideal, and may create more user related problems and nuisance issues than such designs are trying to solve.

In contrast, in the implementation shown in FIGS. 10 to 15, the covers or doors which cover the blood lines or other mechanisms of the blood treatment apparatus are constructed with smart glass, or a smart film, which can change from clear to partially transparent to opaque with the electrical control application.

Referring to FIG. 10, the blood treatment apparatus of this example is a dialysis machine 1000. In contrast with the dialysis machine 100 of the previous examples, the dialysis machine 1000 does not use a blood cassette, although the features of this machine may likewise be employed in cassette-based machines. Here, the blood line set and dialyzer, which are not shown in order to simplify illustration, are attached to the various components on the interior face of the machine 1000 prior to beginning a patient treatment. For example, the blood line set is attached to blood pump rotor 1031 and, depending on treatment mode and blood line configuration, substituate pump rotor 1033 and/or single-needle rotor 1036. A dialyzer holder 1042 is provided for mounting the dialyzer, which is not shown.

The dialysis machine 1000 also includes a monitor 1100 and a base portion 1200, which houses the hydraulics of the machine 1000. The monitor 1100 of this example provides a touchscreen display 1105 for user interface and a pair of indicator lights 1110 and 1115, which are disposed above the display 1105.

Attached to the front of the machine 1000 are a set of doors 1080. In this example, the doors 1080 that cover the blood lines or mechanisms of the dialysis machine are constructed with smart glass, which can change from clear to partially transparent to opaque with the application of an electric circuit. In some examples, flexible films are used to provide the same or analogous function and may provide more design flexibility. Flexible films may provide the same functionality as the smart glass, and may provide additional design options. Examples of smart glass and smart films that may be utilized can be found at https://www.switchglass.com.au, https://www.smarttint.com, and http://www.invisishade.com, although any suitable smart glass, smart film, or other smart material/display technology may be utilized. Such technologies provide for electronically controllable appearance.

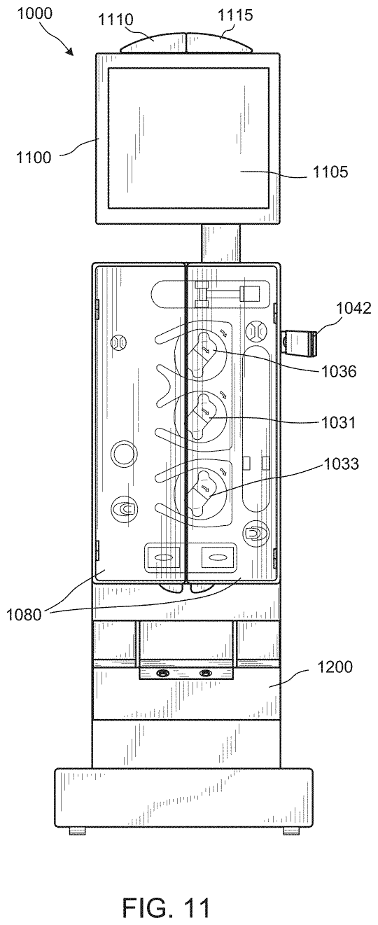

As shown in FIG. 10, the doors are in an open position to allow access to the blood lines and associated components of the machine 1000, for mounting/dismounting of the blood lines and maintenance of the components. FIGS. 11 to 15 show the doors 1080 when closed to cover the mechanisms of the dialysis machine 1000.

In accordance with some examples, during set-up, the doors 1080 on the dialysis machine 1000 are controlled to be transparent, as depicted in FIG. 11. At the beginning of treatment, the doors 1080 can remain transparent (e.g., clear) so the patient or caregiver can see the mechanisms behind the doors 1080 and be assured that the process is proceeding as planned. After a pre-set period of time, or at the patient's direction, or any other suitable triggering, the doors can change from clear to a higher degree of opacity (such as translucent or fully opaque), which may be defined by the patient or pre-set/determined by the manufacturer or software running on the machine 1000 (e.g., on a computer system 3000 described below in connection with FIG. 18). In some examples, the opacity can range from 20% to 100% in the higher-opacity state. Providing the capability to change opacity provides the patient with the benefits of complete transparency to the dialysis process, but also the ability to hide at least some of the “clinical” or “hospital-like” appearance aspects of the machine 1000.

In some examples, during warning or alarm conditions, the dialysis machine 1000 is pre-programmed (e.g., via computer system 3000 described below) to revert the covers or doors 1080 to transparent, so the patient or caregiver can have immediate visibility to the mechanism of the machine 1000 to check for blood leaks, pump malfunction, or other problems.

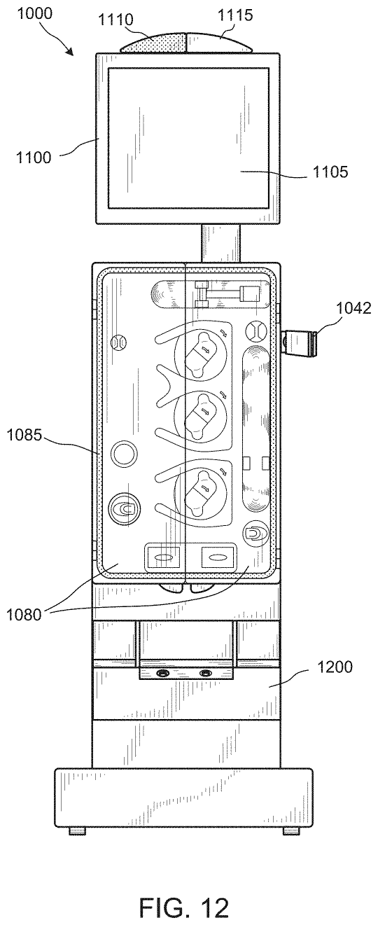

The illustrated example also includes electro-luminescent (EL) wire 1085, which allows indication of an alarm condition to be integrated into the door, (or cover or other part of the machine in accordance with other examples), making the visual alarm more easier to quickly identify. The EL wire 1085 is shown in an illuminated state in FIGS. 12, 14, and 15. Although this example also retains indicator lights 1110 and 1115, in other examples the indicator lights 1110 and 1115 are eliminated, since the EL wire could handle the same functionality. In some examples, there are different colors of EL wire to indicate different conditions. For example, there may be green, yellow, and red EL wires, where green illumination indicates that the machine is operating properly, yellow illumination indicates an alarm condition, and red illumination indicates a severe alarm condition. Illuminating the full array of lights or some combination of them for maximum lighting effect over a variable period of time may also be useful for alerting the user in a darkened room for a more gentle wake-up.

The EL wire may be formed of a phosphor coating between a conductive core (e.g., copper) and an outer conductor (e.g. wrapped wire, such as copper). Applied current causes the phosphor to illuminate. Other examples may use strips of EL panel which can allow for increased width of the illuminated line. Such panels may be formed in a manner similar to EL wire, but with the phosphor between a conductive substrate and an outer conductor (e.g., a clear electrode layer).

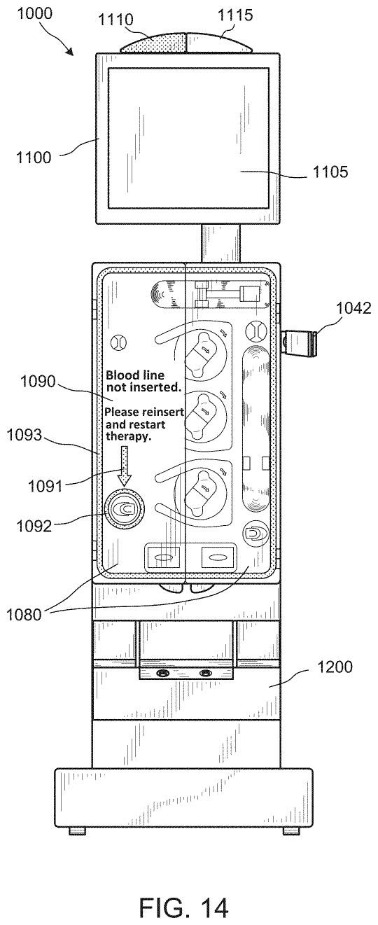

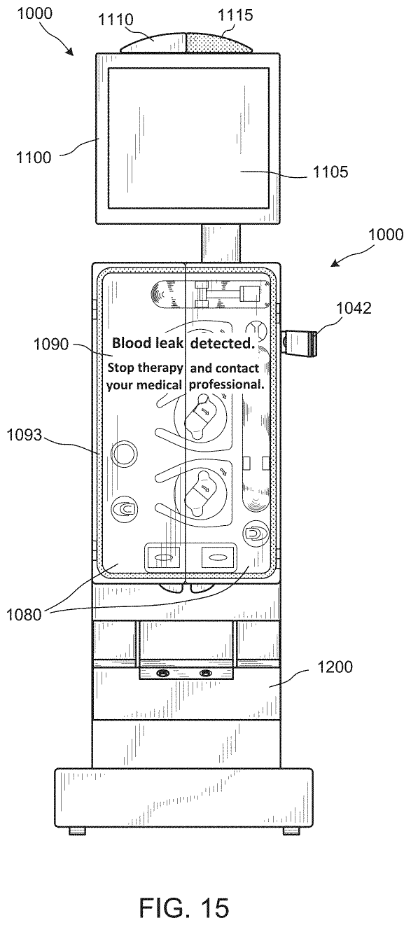

Referring to FIGS. 14 and 15, the machine 1000 can further include a transparent electronic display 1090 capable of displaying messages and/or graphics. The display 1090 may be, for example, super transparent LED glass (see, e.g., http://polytronglass.com/) incorporated into the cover/doors 1080. Software prompts can cause the display 1090 to convey specific information regarding the operation of the machine 1000. For example, during an alarm condition, the display could help direct the patient or caregiver to a specific region of the machine and include on-site trouble shooting information, such as shown in FIG. 14. Here, the machine has sensed that a blood line is not inserted and accordingly operates the display 1090 to show text indicating that the blood line is not inserted and instructing the patient or caregiver to reinsert the blood line and restart therapy. Further, the graphic shown by the display 1090 includes an arrow 1091 and a circle 1092 to highlight the location on the face of the machine 1000 where the action (here, reinsertion of the blood line) is to occur. FIG. 15 illustrates an alarm state whereby the display 1090 indicates that the machine 1000 has detected a blood leak and instructs the patient or operator to contact a medical professional. The display also shows a border illumination 1093 in both of FIGS. 14 and 15.

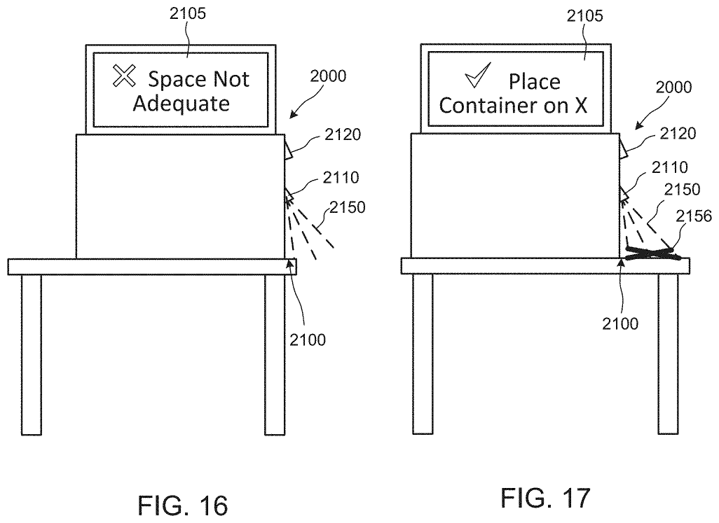

Referring to FIGS. 16 and 17, further incorporation of lighting, e.g., LEDs, as prompts could also be applied for equipment setup (e.g., setup of dialysis or non-dialysis medical equipment) that includes placing certain supplies or containers on a surface 2100 next to a machine 2000. In this example, the machine 2000 includes a light source 2110 that projects light 2150 to the designated location for the supply or containers on a support surface 2100.

The machine 2000 further includes a camera 2120 to detect the portion of projected light 2150 that bounces back to the camera. Referring to FIG. 16, if the light does not adequately reflect back, or fails to match a predetermined expected reflected image, the machine 2000 issues an alert to the person setting up the machine that there is not enough space available for proper setup, since the pattern of the light 2150 corresponds to the space required for the supplies or equipment that need to be positioned next to the machine 2000. In this example, the alert is a visual “x” and a text message displayed on a display screen 2105 of the machine 2000.

Referring to FIG. 17, when the camera detects the projected light 2150 bouncing adequately bouncing back to the camera and/or sufficiently matching an expected reflected image, the machine 2000 recognizes that adequate space is available for the supplies or equipment for a treatment setup. At this stage, the machine 1000 displays an alert on display screen 2105 to indicate that there is sufficient space. Here, the alert is in the form of a check mark and text instructing to place the container/supplies on the “x” pattern 2156 projected by the light source 2110 onto the surface 2100. Although this example utilizes an x-shaped pattern 2156, any suitable pattern (e.g., one or more rectangles, circles, ovals dots, and/or any other regular or irregular shapes or symbols) may be used to indicate the location. This projection may also include a laser keyboard/keypad for quick input of treatment information when a full user interface display is not required or available.

As a further visual alert or indicator, the machine 2000 may control the visible color of the projected light itself. For example, the projected light 2150 may be visible as red whenever adequate space has not been determined, such as in FIG. 16, and green whenever adequate space has been determined, as in FIG. 17.

Further regarding analysis of the image received by the camera 2120, in some examples, the camera 2120 is set to have a focus range corresponding to the expected distance to the surface 2100. Accordingly, the machine 2000 may analyze the sharpness of the reflected image (alone, or in combination with the shape and/or intensity of the reflected image) to determine that adequate space is available in the desired location.

While other machines may require a tray or other platform integrated into the machine itself, the example machine 1000 of FIGS. 16 and 17 may allow omission of such integrated features, since the light and camera system would ensure an adequate “virtual tray.” This may allow the machine 1000 to be smaller in size, and therefore more portable.

FIG. 18 is a block diagram of an example computer system 3000 illustrated in connection with the machines 100, 1000, 2000. That is, the computer system 3000 is illustrative of computer systems that may be incorporated into machines 100, 1000, 2000 described above, or any other implementations of the concepts set forth herein. The system 3000 includes a processor 3010, a memory 3020, a storage device 3030, and an input/output device 3040. Each of the components 3010, 3020, 3030, and 3040 can be interconnected, for example, using a system bus 3050. The processor 3010 is capable of processing instructions for execution within the system 3000. The processor 3010 can be a single-threaded processor, a multi-threaded processor, and/or other processor. Although “processor” may be used as a singular noun herein, including the claims, it should be understood that this term also encompasses multiple physical processors acting in coordination to perform the functions described. The processor 3010 is capable of processing instructions stored in the memory 3020 or on the storage device 3030. The memory 3020 stores information within the system 200. In some implementations, the memory 3020 is a computer-readable medium. The memory 3020 can, for example, be a volatile memory unit or a non-volatile memory unit. The processor 3010 executes the instructions to perform the various control functions described above (e.g., controlling opacity of covers/doors, controlling lights, analyzing camera images, providing alerts, etc.).

The storage device 3030 is capable of providing mass storage for the system 3000. In some implementations, the storage device 3030 is a non-transitory computer-readable medium. The storage device 3030 can include, for example, a hard disk device, an optical disk device, a solid-state drive, a flash drive, magnetic tape, or some other large capacity storage device. The storage device 3030 may alternatively be a cloud storage device, e.g., a logical storage device including multiple physical storage devices distributed on a network and accessed using a network. In some implementations, the information stored on the memory 3020 can also or instead be stored on the storage device 3030.

The input/output device 3040 provides input/output operations for the system 3000. In some implementations, the input/output device 3040 includes one or more of network interface devices (e.g., an Ethernet card), a serial communication device (e.g., an RS-232 10 port), and/or a wireless interface device (e.g., a short-range wireless communication device, an 802.11 card, a wireless modem (3G, 4G, 5G)). In some implementations, the input/output device 3040 includes driver devices configured to receive input data and send output data to other input/output devices, e.g., a keyboard, a printer, and display devices (such as the various controllable visual components described herein). In some implementations, mobile computing devices, mobile communication devices, and other devices are used.

In some implementations, the computer system 3000 is a microcontroller. A microcontroller is a device that contains multiple elements of a computer system in a single electronics package. For example, the single electronics package could contain the processor 3010, the memory 3020, the storage device 3030, and input/output devices 3040.

As used herein, an element or operation recited in the singular and preceded with the word “a” or “an” should be understood as not excluding plural elements or operations, unless such exclusion is explicitly recited. References to “one” embodiment or implementation of the present disclosure are not intended to be interpreted as excluding the existence of additional embodiments that also incorporate the recited features. Furthermore, a description or recitation in the general form of “at least one of [a], [b] or [c],” or equivalent thereof, should be generally construed to include [a] alone, [b] alone, [c] alone, or any combination of [a], [b] and [c].

Implementations of the invention will be apparent to those skilled in the art from a consideration of the specification or practice of the invention disclosed herein. It is intended that the specification and examples be considered as exemplary only, with the true scope and spirit of the invention being indicated by the following claims.

What is claimed is:

1. A medical device, comprising:
a housing;
a plurality of attachment points disposed on a face of the housing and configured to hold a medical fluid tubing system;
a lighting system disposed at or below the face of the housing; and
a processor configured to control the lighting system to (a) visually indicate the locations of the attachment points and (b) in response to a feedback signal indicating whether or not the medical fluid tubing system is attached to at least one of the attachment points, alter the visual indication of the lighting system.

2. The medical device of claim 1, wherein the medical device is a dialysis machine.

3. The medical device of claim 1, wherein the lighting system includes a plurality of light-emitting diodes disposed below the face of the housing.

4. The medical device of claim 3, wherein the face of the housing comprises a translucent material configured to diffuse light emitted from the array of light-emitting diodes.

5. The medical device of claim 1, wherein the feedback signal corresponds to a query response from a user of the medical device.

6. The medical device of claim 1, further comprising a sensor system configured to generate the feedback signal using a plurality of sensors configured to monitor respective attachment points of the plurality of attachment points.

7. The medical device of claim 6, wherein the processor is configured to (a) determine, based on signals from the sensor system, that the medical fluid tubing system has been unseated or dismounted from one of the attachment points and (b) control the lighting system to visually indicate the location of the attachment point at which the unseating or dismounting has been determined.

8. The medical device of claim 7, wherein the processor is configured to cause one or more lights of the lighting system to illuminate in proximity to the location of the attachment point at which the unseating or dismounting has been determined.

9. The medical device of claim 1, wherein the processor is configured to illuminate portions of the lighting system based on the feedback signal and at least one of (a) a predetermined installation sequence and (b) a predetermined dismounting sequence.

10. The medical device of claim 9, wherein the predetermined installation sequence or the predetermined dismounting sequence includes a predetermined order of attachment points to which the medical fluid tubing system is to be mounted or dismounted.

11. The medical device of claim 10, wherein the processor is further configured to:
illuminate a first portion of the lighting system in proximity to a first attachment point; and
responsive to to the feedback signal indicating attachment of the medical fluid tubing system to the first attachment point, illuminating a second portion of the lighting system in proximity to a second attachment point, the second attachment point immediately following the first attachment point in the predetermined order of attachment points.

12. The medical device of claim 11, wherein the processor is further configured to, responsive to to the feedback signal indicating attachment of the medical fluid tubing system to the second attachment point, illuminate a third portion of the lighting system in proximity to a third attachment point, the third attachment point immediately following the second attachment point in the predetermined order of attachment points.

13. The medical device of claim 1, wherein the processor is further configured to:
simultaneously illuminate the lights in proximity to each of the attachment points; and
responsive to the feedback signal indicating attachment of the medical fluid tubing system to a first one of the attachment points, change the appearance of a portion of the lighting system in proximity to the first one of the attachment point.

14. The medical device of claim 1, wherein the altering of the visual indication includes controlling a portion of the lighting system by at least one of: (a) changing light intensity of the portion, (b) powering off the portion, (c) changing the portion from a visually perceptible blinking state to a non-blinking state, and (d) changing a color of the portion.

15. The medical device of claim 14, wherein the portion of the lighting system is in proximity to at least one attachment point where attachment of the tubing system has been detected, and the controlling is in response to the detection.

16. A medical device, comprising:
a housing configured to receive a medical fluid tubing set;
a cover having an open position to provide user access to install and remove the blood tubing set, and a closed position to at least partially cover the blood tubing set; and
a processor configured control the appearance of the cover based on a sensed condition of the device.

17. The medical device of claim 16, wherein the medical device is a dialysis machine, and medical fluid tubing set is a blood tubing set.

18. The medical device of claim 16, wherein the processor is configured to change the appearance of the cover from a first state, in which the cover is transparent, to a second state, in which the cover is translucent or opaque.

19. The medical device of claim 18, wherein:
the sensed condition is whether the cover is in the open position or the closed position; and
the processor is configured to change the appearance of the cover from the first state to the second state in response to a determination that the cover in the closed position.

20. The medical device of claim 19, wherein the processor is configured to wait a period of time after sensing the closed position before changing the appearance from the first state to the second state.

21. The medical device of claim 18, wherein the processor is configured to change the appearance of the cover from the second state to the first state in response to an alarm condition of the blood treatment device.

22. The medical device of claim 16, wherein the cover includes a transparent electronic display.

23. The medical device of claim 22, wherein the transparent electronic display is a super transparent LED glass.

24. The medical device of claim 22, wherein the processor is configured to, in response to a determination of an alarm condition, display an alarm message on the cover.

25. The medical device of claim 16, wherein the cover includes at least one hinged door.

26. A medical device, comprising:
a housing;
a light source configured to project light onto a surface adjacent to the housing;
an image sensor configured to sense reflected portions of light projected by the light source; and
a processor configured to (a) determine, based on the reflected portions of light sensed by the image sensor, whether the surface adjacent to the housing satisfies a predetermined size requirement, and (b) communicate the determination to a user of the device.

27. The medical device of claim 26, wherein the medical device is a dialysis machine.

28. The medical device of claim 26, further comprising a display screen, wherein the processor is configured to, responsive to a determination that the surface satisfies the predetermined size requirement, control the display screen to display a message instructing the user to place an item on the surface.

29. The medical device of claim 28, wherein the message includes an instruction to place the item on a visual indicator projected onto the surface by the light source.

30. The medical device of claim 26, wherein the processor is configured to change the light projected by the light source from a first visible color to a second color in response to a determination that the surface satisfies the predetermined size requirement.
